(19) United States
(12) Patent Application Publication
(10) Pub. No.: US 2008/0166243 A1
(43) Pub. Date: Jul. 10, 2008

(54) Liquid for Compressing a Gaseous Medium and Use of the Same

(76) Inventors: Michael Kotschan, Leoben (AT); Roland Kalb, Leoben (AT)

Correspondence Address: OSTROLENK FABER GERB & SOFFEN, 1180 AVENUE OF THE AMERICAS, NEW YORK, NY, 100368403, US

(21) Appl. No.: 11/913,740

(22) PCT Filed: May 3, 2006
(86) PCT No.: PCT/EP2006/062009

§ 371 Date: Nov. 6, 2007

(30) Foreign Application Priority Data
May 6, 2005 (AT) A 779/2005

(51) Int. Cl. C07C 211/62; C07F 9/54; C07C 381/12; C07D 233/54; F04F 11/00

(52) U.S. Cl. 417/92; 564/281; 568/9; 568/18; 548/342.1; 548/336.1

(57) ABSTRACT

The invention relates to a liquid for compressing a gaseous medium which transfers the force required for compression directly to the gas. The liquid has a vapour pressure of less than 10⁻³ mbar.

### CROSS REFERENCE TO RELATED APPLICATION

The present application is a 35 U.S.C. §371 national phase conversion of PCT/EP2006/062009 filed May 3, 2006, which claims priority of Austrian Application No. A779/2005, filed May 6, 2005 which are incorporated herein in their entirety.

### BACKGROUND OF THE INVENTION

1. Field of the Invention

The invention relates to a liquid for the compression of a gaseous medium which transfers the force required for compression directly to the gas.

2. Background Art

At the present time, in known methods and devices for compressing a gaseous medium, piston compressors or piston compressor systems are usually used. Piston compressors require corresponding sealing systems to separate the medium to be compressed from the medium driving the piston, for example, hydraulic oil.

For example, custom-fit cylinders with pistons and correspondingly effective dynamic sealing systems are required for the compression of hydrogen, natural gas and highly pure media and these usually incur high production and maintenance costs.

In a German patent application having the official file reference 102004046316.6, which has not yet been published, a method and a device for compressing a gaseous medium are described in which the compression of the gaseous medium is effected by a liquid in which the gaseous medium does not dissolve and/or which can be separated from the gaseous medium without any residue.

### SUMMARY OF THE INVENTION

The object of the present invention is to provide a liquid for compressing a gaseous medium which is particularly suitable as an operating means for transferring the force required for the compression directly to the gas. In addition, the liquid for compression of the gaseous medium should also be suitable for being used in conventional compactors and compressors.

This object is achieved according to the invention whereby the liquid has a vapour pressure of less than 10⁻³ mbar.

According to a preferred embodiment of the invention, the liquid has a vapour pressure of less than 10⁻⁶ mbar, in particular less than 10⁻⁹ mbar.

A liquid having such a low vapour pressure is particularly suitable for being used as a “liquid compressor piston” since entrainment of liquid into the gas to be compressed through vaporisation is avoided. Low flammability, low compressibility and good conductivity are advantageously associated with the low vapour pressure.

Those liquids which are thermally stable in the temperature and pressure range of the compression process and which have no solubility or only very low solubility for the gas to be compressed are particularly suitable for the compression of the gaseous medium within the scope of the invention.

Particularly suitable are liquids which are ionic compounds or a mixture of a plurality of ionic compounds having the general formula [Q⁺]ₙ[Zⁿ⁻],

wherein the relevant cation [Q⁺] is a quarternated or protonated ammonium [R¹R²R³R⁴N⁺], phosphonium [R¹R²R³R⁴P⁺] or sulfonium [R¹R²R³S⁺] cation or a similar quaternated or protonated nitrogen, phosphorus or sulphur heteroaromatic compound,

wherein the groups R¹, R², R³, R⁴ are all the same, partly the same or different,

wherein the groups R¹, R², R³ and R⁴ are protons (hydrogen atoms), linear, cyclic, branched, saturated or unsaturated alkyl groups, mono- or polycyclic aromatic or heteroaromatic groups or derivatives of these groups substituted with other functional groups,

wherein R¹, R², R³, R⁴ can also be connected to one another (multibond groups),

wherein [Zⁿ⁻] is an anion having a negative valence number n⁻ or also is an anion protonated with m hydrogen atoms [HₘZ^{(n-m)-}].

Precisely these ionic compounds have those properties—thermal stability in the temperature and pressure range of the component and no solubility or very low solubility for the gaseous medium—which make them ideal for the compression of gaseous media.

In this connection, reference is made to WO 2005/021484 A2 which is concerned with a method for producing ionic liquids, ionic solids or mixtures of the same. The ionic compounds used within the scope of the subject invention can be produced efficiently and cost-effectively using the method described in this document.

In a preferred embodiment of the invention, [Zⁿ⁻] can be an anion which forms an ionic compound with [Q⁺] suitable for compressing a gaseous medium.

Alternatively to this, [Zⁿ⁻] can be one of the following anions: tetrafluoroborate [BF₄⁻], hexafluorophosphate [PF₆⁻], bis-(trifluoromethylsulfonyl)imide [(CF₃SO₂)₂N⁻], tris-(trifluoromethylsulfonyl)methide [(CF₃SO₂)₃C⁻], trifluoromethane sulfonate [CF₃SO₂], trifluoroacetate [CF₃CO₃⁻], methane sulfonate [CH₃SO₂⁻], dicyanamide [NCNCN⁻], tricyanamide[C(CN)₃⁻], formiate, propanoate, butanoate, heptafluorobutanoate, pentanoate, hexanoate, heptanoate, octanoate, nonanoate, decanoate, ethane sulfonate, propane sulfonate, butane sulfonate, pentane sulfonate, hexane sulfonate, heptane sulfonate, octane sulfonate, nonane sulfonate, decane sulfonate, acetate, chloride, bromide, iodide, nitrate, chlorate, perchlorate, bromate, perbromate, phosphate, borate, sulfate, hydrogen sulfate, alkyl sulfate, arylsulfate, dialkylphosphate, diarylphosphate, alkylphosphonate, arylphosphonate, alkylcarboxylate, arylcarboxylate, carbonate, hydrogen carbonate, alkylcarbonate, arylcarbonate, gluconate, tartrate, ascorbate, lactate, citrate, benzoate, salicylate etc. or a derivative of the same.

Of the ionic compounds, the following also in combination with one another, are particularly suitable: 1-ethyl-3-methylimidazolium ethylsulfate (CAS 342573-75-5) or 1-butyl-3-methylimidazolium methylsulfate (CAS 401788-98-5) or 1-ethyl-3-methylimidazolium thiocyanate (CAS 331717-63-6) or 1-butyl-3-methylimidazolium thiocyanate (CAS 344790-87-0) or methyltributylammonium dibutylphosphate.

Certain molecular liquids also have a low vapour pressure of the order of magnitude of 10⁻⁴ to 10⁻¹⁰. Particularly suitable from the class of these liquids are a perfluoropolyether, a polyphenyl ether or polyphenyl thioether, a silicone oil, a mineral oil, a synthetic oil or a mixture of at least two of these substances.

Liquids comprising a mixture of at least one ionic compound with at least one molecular liquid can also be used within the scope of the invention.

Most of the particularly suitable liquids mentioned within the scope of the invention for compressing a gaseous medium already have favourable lubrication, corrosion and sealing properties. However, for certain applications and also when using certain substances, it can be advantageous to add ionic or molecular additives to the substance or the liquid such as for example, corrosion protection additives, oxidation protective additives, reduction protection additives, pH buffer substances and/or acid interceptors, complex forming agents, emulsifiers, dispersants, detergents, wear protection additives, extreme pressure additives, friction modifiers, viscosity modifiers, gelling agents, sealing additives, preservatives, pour-point additives, foam inhibitors, radical interceptors, water regulators.

According to a preferred embodiment, the liquid is an emulsion and according to another preferred embodiment, the liquid can be liquid-crystalline or a mixture of a micro- to nanocrystalline solid with at least one ionic and/or at least one molecular liquid. In some of their characteristic values, such liquids tend to behave as solids which makes them particularly suitable for use in compressing gaseous media.

Liquids proposed according to the invention are preferably used in a compactor or compressor system for compressing a gaseous medium. In this case, the liquid can form the phase boundary directly with the gaseous medium to be compressed or a membrane can be incorporated at its phase boundary with the gas to be compressed.

Depending on their other properties, the liquid used is at the same time advantageously suitable as means for operating valves, measuring, regulating and control units, cooling system, moving mechanical parts or seals of the compactor or compressor system.

The liquid used can furthermore be liquid under the prevailing or provided operating conditions but can be present in the solid state of aggregation before starting up the compactor or compressor. In this way, an ionic solid present in the solid state of aggregation before starting the compactor or compressor can also be used.

Further features, advantages and details of the invention are obtained from the following description.

### DETAILED DESCRIPTION OF EMBODIMENTS OF THE INVENTION

Some of the terms used are to be understood as follows:

Ionic liquids: ionic liquids are—in the sense of the acknowledged literature (e.g. Wasserscheid, Peter; Welton, Tom (Eds.); “Ionic Liquids in Synthesis”, Verlag Wiley-VCH 2003; ISBN 3-527-30515-7; Rogers, Robin D.; Seddon, Kenneth R. (Eds.); “Ionic Liquids—Industrial Applications to Green Chemistry”, ACS Symposium Series 818, 2002; ISBN 0841237891)—liquid organic salts or mixtures of salts consisting of organic cations and organic or inorganic anions, having melting points below 100° C.

Ionic solids: in this application the term “ionic solids” includes salts in the sense of ionic liquids, having melting points of 100° C. or higher. Apart from this melting point range specified by definition and the associated state of aggregation, there is no fundamental chemical or physical difference from ionic liquids.

Ionic compounds: are all ionic liquids and ionic solids in the sense of the definitions given above.

Molecular liquids: unlike ionic compounds, are conventional liquids constructed on a molecular basis; they are therefore not constructed of ions, that is not salts.

Ionic compounds—compounds which are ionic liquids or ionic solids—have extremely interesting properties such as, for example, a very low to almost barely measurable vapour pressure, a very large liquidus range, good electrical conductivity and unusual solvation properties. These properties make these compounds ideal for use in various areas of technical applications. For example, they can be used as solvents (in organic and inorganic synthesis in general, in the analysis of transition metals, biocatalysis, phase transfer catalysis, in multiphase reactions, in photochemistry, in polymer synthesis and in nanotechnology), as extractants (in liquid-liquid and liquid-gas extraction in general, desulphurisation of crude oil, removal of heavy metals from waste water, liquid membrane extraction), as electrolytes (in batteries, fuel cells, capacitors, solar cells, sensors, in electrochemistry, electroplating technology, in electrochemical metal processing, in electrochemical synthesis in general, in electro-organic synthesis, nanotechnology), as lubricants, as thermofluids, as gels, as reagents for organic synthesis, in “green chemistry” (replacements for volatile organic compounds), as anti-statics, in special applications in analysis (gas chromatography, mass spectroscopy, capillary zone electrophoresis), as liquid crystals etc. (this list is not exhaustive). In this respect, reference is made for example to “Rogers, Robin D.; Seddon, Kenneth R. (Eds.); Ionic Liquids—Industrial Applications to Green Chemistry, ACS Symposium Series 818, 2002; ISBN 0841237891” and “Wasserscheid, Peter; Welton, Tom (Eds.); Ionic Liquids in Synthesis, Verlag Wiley-VCH 2003; ISBN 3527305157”.

The properties can be optimised within wide limits for the respective application by varying the structure of anion and cation or varying the combination thereof, which has led to the introduction of the term “designer solvents” for ionic liquids (see, for example, Freemantle, M.; Chem. Eng. News, 78, 2000, 37).

Particularly suitable ionic compounds within the scope of the invention include low-melting organic salts consisting of organic cations and organic or inorganic anions having the general formula [Q⁺]ₙ[Zⁿ⁻],

wherein the relevant cation [Q⁺] is a quarternated or protonated ammonium [R¹R²R³R⁴N⁺], phosphonium [R¹R²R³R⁴R⁴P⁺] or sulfonium [R¹R²R³S⁺] cation or a similar quaternated or protonated nitrogen, phosphorus or sulphur heteroaromatic compound see FIG. 1),

wherein the groups R¹, R², R³, R⁴ can be all the same, partly the same or different. These groups R¹, R², R³, R⁴ can be protons (hydrogen atoms), linear, cyclic, branched, saturated or unsaturated alkyl groups, mono- or polycyclic aromatic or heteroaromatic groups or derivatives of these groups substituted with other functional groups, wherein R¹, R², R³, R⁴ can also be connected to one another (multibond groups),

wherein [Zⁿ⁻] is an anion having a negative valence number n⁻.

[Zⁿ⁻] can also be an anion protonated with m hydrogen atoms

[HₘZ^{(n-m)-}]. In the case of phosphate [PO₄³⁻], this would be, for example, the dihydrogen phosphate [H₂PO₄⁻] and the hydrogen phosphate [HPO₄²⁻]). In principle, [Zⁿ⁻] can be an anion which forms an ionic compound with [Q⁺] suitable for compressing a gaseous medium.

Preferably [Zⁿ⁻] is one of the following anions: tetrafluoroborate [BF₄⁻], hexafluorophosphate [PF₆⁻], bis-(trifluoromethylsulfonyl)imide [(CF₃SO₂)₂N⁻], tris-(trifluoromethylsulfonyl)methide [(CF₃SO₂)₃C⁻], trifluoromethane sulfonate [CF₃SO₂], trifluoroacetate [CF₃CO₃⁻], methane sulfonate [CH₃SO₂⁻], dicyanamide [NCNCN⁻], tricyanamide[C(CN)₃⁻], formiate, propanoate, butanoate, heptafluorobutanoate, pentanoate, hexanoate, heptanoate, octanoate, nonanoate, decanoate, ethane sulfonate, propane sulfonate, butane sulfonate, pentane sulfonate, hexane sulfonate, heptane sulfonate, octane sulfonate, nonane sulfonate, decane sulfonate, acetate, chloride, bromide, iodide, nitrate, chlorate, perchlorate, bromate, perbromate, phosphate, borate, sulfate, hydrogen sulfate, alkyl sulfate, arylsulfate, dialkylphosphate, diarylphosphate, alkylphosphonate, arylphosphonate, alkylcarboxylate, arylcarboxylate, carbonate, hydrogen carbonate, alkylcarbonate, arylcarbonate, gluconate, tartrate, ascorbate, lactate, citrate, benzoate, salicylate etc. or a derivative of the same.

The following FIG. 1 already mentioned above gives examples of heteroalicyclic and heteroaromatic cations [Q⁺], this being an incomplete listing:

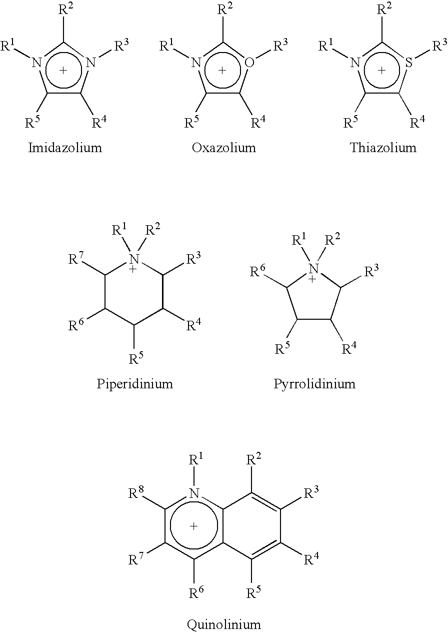

### FIG. 1

Note: For the additional groups R⁵, R⁶, R⁷, R⁸, the same definition applies as for the described definition of the groups R¹, R², R³, R⁴.

Ionic compounds are particularly well suited as a medium or liquid for the compression of a gaseous medium, in particular on account of their extremely low, as far as immeasurable, vapour pressure. A low vapour pressure is to be understood in this case as a vapour pressure lower than 10⁻³ bar, in particular lower than 10⁻⁶ or even lower than 10⁻⁹ mbar. The low vapour pressure is responsible for effectively preventing any undesirable entrainment of the liquid or ionic compound used for compression of the gaseous medium as a result of vaporisation. The extremely low vapour pressure is associated with a very low flammability and a low compressibility. A further advantageous property of ionic compounds is their low tendency to electrostatic charging, thereby avoiding any potential ignition of combustible gases. A plurality of gases such as for example, hydrogen and carbon monoxide have a very low solubility in many ionic compounds, thereby minimising any foam formation and compression losses. A further advantage is the good adaptability of the chemical and physical properties of ionic compounds by suitable variation of the structure of cations and anions and their combination, in the attainable high stability with respect to thermal loads and chemical influences. High decomposition temperatures and a good redox stability can be obtained.

Some ionic liquids exhibit liquid-crystalline properties and thus behave more like solids than like liquids in some of their characteristics, which particularly recommends them for an application as “liquid compressor pistons”.

Particularly suitable ionic liquids having low vapour pressure for the compression of a gaseous medium within the scope of the invention include: 1-ethyl-3-methylimidazolium ethylsulfate (CAS 342573-75-5) or 1-butyl-3-methylimidazolium methylsulfate (CAS 401788-98-5) or 1-ethyl-3-methylimidazolium thiocyanate (CAS 331717-63-6) or 1-butyl-3-methylimidazolium thiocyanate (CAS 344790-87-0) or methyltributylammonium dibutylphosphate. Mixtures of at least two of these substances can also advantageously be used.

Possible alternatives to ionic liquids for the compression of gaseous media within the scope of the invention are other liquids which also have a low vapour pressure, albeit somewhat higher than most ionic compounds. Particularly suitable among the molecular liquids, for example are: perfluoropolyether (e.g. Fombline®/Solvay Solexis Inc.) or polyphenyl ether or polyphenyl thioether (e.g. Santovac®/Monsanto Co.) or a silicone oil (e.g. a tetramethyltetraphenyltrisiloxane AN140/Wacker Chemie GmbH) or a mineral oil (e.g. Balzers B3/Balzers AG) or synthetic oil (e.g. Alcatel 111/Alcatel Hochvakuum Technik GmbH) or any other liquid which is characterised by a low to very low vapour pressure and which has suitable physical-chemical properties for the said application such as, for example, low gas solubility, thermal and chemical stability, suitable liquidus range etc. (this list is not exhaustive).

The ionic compounds and the molecular liquids can additionally be mixed with ionic and/or molecular additives in order to optimise further requirements such as, for example, corrosion properties, sealing properties, viscosity, tribological behaviour, chemical and physical stability, compatibility with respect to construction materials such as for example, metals and elastomers. At least one of the following additives can be considered in particular: corrosion protection additives, oxidation protective additives, reduction protection additives, pH buffer substances and/or acid interceptors, complex forming agents, emulsifiers, dispersants, detergents, wear protection additives, extreme pressure additives, friction modifiers, viscosity modifiers, gelling agents, sealing additives, preservatives, pour-point additives, foam inhibitors, radical interceptors, water regulators.

Liquids which can be used according to the invention can also be mixtures of at least one ionic compound with at least one molecular liquid. In this case, the liquid can be an emulsion or liquid-crystalline or a mixture of a micro- to nanocrystalline solid with at least one ionic and/or at least one molecular liquid.

In addition to the compression of the gaseous medium, the liquids covered by the invention can furthermore take over other tasks in a compactor or compressor system: they can be used at the same time as operating means in all assemblies and components pertaining to the compressor system such as for example, valves, measuring, regulating and control units, cooling systems, moving mechanical parts such as for example bearings and sealing systems.

In addition, certain liquids which can be used within the scope of the invention can remove solid, liquid or gaseous impurities which may be present in the gas to be compressed such as for example water, oxygen, solvent residues.

The liquid according to the invention used in a compactor or compressor system can at the same time directly form the phase boundary with the gaseous medium to be compressed or alternatively to this, a membrane can be incorporated at the phase boundary with the gas to be compressed.

Before use as a compressing agent, the ionic compound can also be an ionic solid which is then liquid in the typical temperature range used.

Although the present invention has been described in relation to particular embodiments thereof, many other variations and modifications and other uses will become apparent to those skilled in the art. Therefore, the present invention is not limited by the specific disclosure herein.

1-19. (canceled)

20. A method for compressing a gaseous medium, which method comprises contacting the gaseous medium with a liquid adapted to transfer a compressing force directly to said gaseous medium and applying a compressing force to said gaseous medium through said liquid, said liquid comprising a liquid ionic compound or a mixture of a plurality of said liquid ionic compounds, each said liquid ionic compound having the general formula [Q⁺]ₙ[Zⁿ⁻],
wherein the cation [Q⁺] is a quarternated or protonated ammonium [R¹R²R³R⁴N⁺], phosphonium [R¹R²R³R⁴P⁺] or sulfonium [R¹R²R³S⁺] cation or a similar quaternated or protonated nitrogen, phosphorus or sulphur heteroaromatic compound,
wherein the groups R¹, R², R³, R⁴ are all the same, partly the same or different,
wherein the groups R¹, R², R³, R⁴ are protons (hydrogen atoms), linear, cyclic, branched, saturated or unsaturated alkyl groups, mono- or polycyclic aromatic or heteroaromatic groups or derivatives of these groups substituted with other functional groups,
wherein R¹, R², R³, R⁴ can also be connected to one another (multibond groups),
wherein [Zⁿ⁻] is an anion having a negative valence number n⁻ or also
is at least one of an anion protonated with m hydrogen atoms [HₘZ^{(n-m)-}], and a molecular liquid for compression of a gaseous medium wherein the respective liquid transfers the force required for compression directly to the gas and has a vapour pressure of less than 10⁻³ mbar.

21. The method according to claim 20, wherein the liquid has a vapor pressure of less than 10⁻⁶ mbar.

22. The method according to claim 20, wherein the liquid is thermally stable in the temperature and pressure range of the compression process.

23. The method according to claim 20, wherein the liquid has no solubility or only very low solubility for the gas to be compressed.

24. The method according to claim 20, wherein [Zⁿ⁻] is an anion which forms an ionic compound with [Q⁺] suitable for compressing a gaseous medium.

25. The method according to claim 20, wherein [Zⁿ⁻] is selected from the group consisting of tetrafluoroborate [BF₄⁻], hexafluorophosphate [PF₆⁻], bis-(trifluoromethylsulfonyl)imide [(CF₃SO₂)₂N⁻], tris-(trifluoromethylsulfonyl)methide [(CF₃SO₂)₃C⁻], trifluoromethane sulfonate [CF₃SO₂⁻], trifluoroacetate [CF₃CO₃—], methane sulfonate [CH₃SO₂—], dicyanamide [NCNCN^{-], tricyanamide[C(CN)}₃⁻], formiate, propanoate, butanoate, heptafluorobutanoate, pentanoate, hexanoate, heptanoate, octanoate, nonanoate, decanoate, ethane sulfonate, propane sulfonate, butane sulfonate, pentane sulfonate, hexane sulfonate, heptane sulfonate, octane sulfonate, nonane sulfonate, decane sulfonate, acetate, chloride, bromide, iodide, nitrate, chlorate, perchlorate, bromate, perbromate, phosphate, borate, sulfate, hydrogen sulfate, alkyl sulfate, arylsulfate, dialkylphosphate, diarylphosphate, alkylphosphonate, arylphosphonate, alkylcarboxylate, arylcarboxylate, carbonate, hydrogen carbonate, alkylcarbonate, arylcarbonate, gluconate, tartrate, ascorbate, lactate, citrate, benzoate, salicylate and derivatives thereof.

26. The method according to claim 20, wherein the liquid comprises 1-ethyl-3-methylimidazolium ethylsulfate (CAS 342573-75-5) or 1-butyl-3-methylimidazolium methylsulfate (CAS 401788-98-5) or 1-ethyl-3-methylimidazolium thiocyanate (CAS 331717-63-6) or 1-butyl-3-methylimidazolium thiocyanate (CAS 344790-87-0) or methyltributylammonium dibutylphosphate or a mixture of at least two of these substances.

27. The method according to claim 20, wherein the liquid is a perfluoropolyether, a polyphenyl ether or polyphenyl thioether, a silicone oil, a mineral oil, a synthetic oil or a mixture of at least two of these substances.

28. The method according to claim 20, wherein the liquid contains at least one ionic or molecular additive such as, for example, selected from the group consisting of corrosion protection additives, oxidation protective additives, reduction protection additives, pH buffer substances and/or acid interceptors, complex forming agents, emulsifiers, dispersants, detergents, wear protection additives, extreme pressure additives, friction modifiers, viscosity modifiers, gelling agents, sealing additives, preservatives, pour-point additives, foam inhibitors, radical interceptors and water regulators.

29. The method according to claim 20 wherein the liquid is in the form of an emulsion.

30. The method according to claim 20 wherein the liquid is in the form of a liquid-crystalline liquid or wherein the liquid is in a mixture with a micro- to nanocrystalline solid.

31. The method according to claim 20, wherein the liquid forms a phase boundary directly with the gaseous medium to be compressed.

32. The method according to claim 31, wherein a membrane is incorporated at the phase boundary between the liquid and the gas to be compressed.

33. The method according to claim 31, wherein the application of a compressing force through said liquid is additionally used as means for operating valves, measuring, regulating and control units, cooling system, moving mechanical parts or seals of the compactor or compressor system.

34. The method according to claim 33, wherein the liquid is in liquid form under operating conditions and wherein the liquid is present in a solid state aggregation before starting up a compactor or compressor in which said liquid is placed.

35. The method according to claim 21 wherein the liquid has a vapor pressure of less than 10⁻⁹ mbar.
